(19) United States
(12) Patent Application Publication
(10) Pub. No.: US 2013/0018400 A1
(43) Pub. Date: Jan. 17, 2013

(54) STATUS CONTROL FOR ELECTRICALLY POWERED SURGICAL TOOL SYSTEMS

(71) Applicants: Sven Milton, Vikingstad (SE); Anders Möllstam, Saltsjo-boo (SE)

(72) Inventors: Sven Milton, Vikingstad (SE); Anders Möllstam, Saltsjo-boo (SE)

(21) Appl. No.: 13/545,157

(22) Filed: Jul. 10, 2012

Related U.S. Application Data
(60) Provisional application No. 61/572,096, filed on Jul. 11, 2011.

(51) Int. Cl. A61B 17/32

(52) U.S. Cl. 606/167

(57) ABSTRACT

The present invention relates to a method for determining a utilization level (UL; ULₜₒₜ) indicating a degree of utilization of a re-usable cutting accessory (4) for endoscopic surgery, such as an arthroscopic shaver blade, removably connectable to a handpiece (2) comprising a motor (21) driving the cutting accessory (4) during surgical procedures. The method provides a reliable measure of the utilization level of the cutting accessory (4) by determining the utilization level (UL) of the cutting accessory (4) through calculation of at least one utilization factor (UF1, UF2) that is calculated based on a torque (40) measured when said cutting accessory (4) is driven by said motor (21).

### RELATED APPLICATIONS

This application is claiming priority to U.S. Application No. 61/572,096 filed on Jul. 11, 2011 and is incorporated herein by reference in its entirety.

### BACKGROUND OF THE INVENTION

1. Technical Field

The present invention relates to medical devices used in surgery including arthroscopic shavers with integrated identification and status control systems.

2. Description of the Related Art

Endoscopic surgery is performed within the natural cavities of the human body. A small hole is created in the skin of the patient and an optical instrument, the endoscope, is positioned in the cavity. The endoscope can consist of a rigid flexible tube having channels for light, fluid etc., depending on the endoscopy application. Specific applications of this procedure include: Laparoscopy, enteroscopy, colonoscopy, sigmoidoscopy, proctoscopy, cystoscopy, arthroscopy, etc.

The ability to perform endoscopic surgery is enhanced by the development of powered surgical tools. Many of these tools take the form of motorized hand pieces to which a cutting accessory has been attached. For example, arthroscopic shaver blades are specialized medical devices used by surgeons during arthroscopic surgery. The main advantage of said powered surgical tools is their ability to accomplish multiple functions, such as bone removal, and suction with one tool, whereas, their primary drawbacks continue to be the high costs associated with their use.

Reusable medical devices are devices that are designed and labeled for use on multiple patients provided that after each use, an appropriate reprocessing protocol and functionality check is performed. They are made of materials that can withstand repeated reprocessing, such as cleaning, disinfecting, or sterilization.

Consequently, proper design for reusability has been important in the common effort for providing cost effective healthcare. The high cost of disposing of medical waste, increasing biohazardous waste in the environment and burgeoning healthcare costs are the reasons behind the increased demand of reusable devices and devices with extended lifetime. The reusable medical devices and instruments market is a growing industry wherein orthopedic and general surgery instruments and devices account for most of the trade. Basically, reusable supplies are often much more inexpensive than disposable supplies, even after including the cost of cleaning, sterilizing and the labor cost for repackaging and inspecting them.

However, over time and through ordinary usage, reusable surgical instruments suffer wear or damage and eventually reach the end of their life cycle. Thus, it is necessary to have systems periodically accounting for the usage of reusable surgical instruments so that they can be replaced as necessary. The aim is to avoid replacement uniquely based on visual appreciation. This is crucial for efficiency and safety reasons, as well as for environmental and cost-related issues.

Current methods allow users to track, inspect and verify the surgical instrument. For example U.S. Pat. No. 7,837,694 teaches an identification method in which the identification information from a RFID tag is compared with previously stored information about the surgical instrument's service; and in the method it is reprogrammed with the number of times and the dates where servicing has been performed to assess, for example, the surgical instruments usage.

Patent application WO9427516 discloses a method in which wear and the need for service of an arthroscopic shaver or a surgical saw are measured by periodically recording the amount of use of attachments such as a cutting device to the shaver and by determining the number of times the arthroscopic shaver has been subjected to autoclaving or a sterilization procedure. In the case of battery power instruments the amount of battery charge used during a particular operation is also monitored. Similarly, patent application EP1537881 teaches a system enabling the user to track the number of remaining uses of a reusable arthroscopic shaver. The system incorporates sterilization indicator tabs showing the number of times a reusable shaver blade has been used and sterilized.

Other inventions in this field focus on improving safety and avoiding deterioration of the surgical device rather than on detecting actual wear and reacting to it, for example by operating a surgical device at its optimal operational limits i.e. motor speed and torque. U.S. Pat. No. 6,017,354 discloses systems allowing that tools, having specific power and control signal requirements, to receive appropriate energization. The maximum torque and speed the motor should deliver are considered in an attempt to avoid undesirable results related to overheating of the motor or wearing out of its components. These systems neither disclose nor address the problem related to the wear of the reusable accessory. In addition, U.S. Pat. No. 7,758,613 teaches monitoring the torque generated by the motor, but in this case, this factor is used for sensing the physical parameters of the tissue on which the device has to work. U.S. Pat. Nos. 4,705,038, 5,269,794 and 6,846,308 describe arthroscopic systems that detect the surgical device i.e. type of cutting device attached to the handpiece. The motor can thus be operated at the optimal operating speed range for the specific cutting device. A system for a surgical handpiece powering a cutting accessory that is attached to it via a drive coupling is disclosed in patent application U.S. 2003/0093103. Here the identification system includes memories describing the operating characteristics of all components i.e. preferred speed and maximum speed for driving the coupling, maximum motor torque, etc. The afore-mentioned devices operate by reference to information that has been previously loaded onto the memory of either the cutting device or the handpiece and do not consider the surgical device actual operating capabilities. Notwithstanding, there are a couple of systems such as the one described in U.S. Pat. No. 5,400,267, wherein the medical device apart from storing information specific for the medical device i.e. voltage limits, current parameters, model number, etc.; even stores information on the use of the medical device in individual procedures i.e. maximum sterilization count, maximum actuation count, etc.

Likewise, the system taught in patent application U.S. 2009/0292304, also aims to inform the user about a surgical tool's operation parameters. In this case, real-time information on the state of the system is used to update previously loaded information and to regulate operation. In one specific embodiment of that application wear profile is determined by comparing a predetermined “Max Time” use of the cutting accessory with the total time the cutting accessory has been used in different surgical procedures. The Max Time use value is obtained from empirical studies indicating how long a certain accessory can be used before its cutting surfaces become excessively worn to the level at which they may not efficiently cut tissue. Data representative of cumulative watt minutes of power consumed in actuating the cutting accessory are also considered for calculating the total cutting accessory's wear, measured by reduced efficiency. Methods like this in which wear estimations are based on the consumed amount of electrical power or the number of times the shaver has been used assume that there is a lineal relationship between wear and said factors and do not consider the influence of variable actual factors, such as the torque peak effects, tissue properties etc. on the lifetime of the shaver.

In general, the problem of the current systems for reusable shavers are related to the fact that the number of times a cutting accessory e.g. a shaver blade, has been used, measured by the number of times it has been fitted to the handpiece, the power consumed or the number of times it has been sterilized does not well enough reflect the wear of the specific blade i.e. when it has to be replaced. It may have been used in several very short procedures, or in a surgical operation with several high torque peaks, or on demanding tissue, which means very different wear and need for replacement. Furthermore, those systems relying on the information at which the cutting device operates optimally are only aimed at minimizing wear, and as such, do not address the problem of measuring and informing about the device's actual wear. There is still a need for reliable and accurate systems to measure usage or wear of reusable surgical devices based on variable actual factors and on an individual device basis.

### SUMMARY OF THE INVENTION

The invention herein provides a system that overcomes the shortcomings of the prior art by logging and relaying on actual and historical information for a specific surgical device. The Smart Shaver™ system described herein, measures, calculates and monitors the influence of different variable factors on the deterioration of an attachable cutting accessory and logs this specific information with each device. These factors include but are not limited to, the cutting accessory's accumulated rotations, the motor actually measured accumulated torque and torque peaks over a set value and the number of times and the duration in which the motor drives the cutting accessory above pre-defined accumulated torque values, and number of sterilizations. Consequently, we provide the user with accurate and reliable information on efficiency and actual wear/usage of a powered surgical tool's reusable accessory e.g. the blade of an arthroscopic shaver. This is very important for ensuring patient safety and fulfilling legal requirements on surgical power tools. On the other hand, it allows a cost-effective use of the cutting accessory throughout its entire life cycle.

In preferred embodiments the usage information may be employed to operate the surgical device to prevent further operation of the system with an excessively worn cutting accessory and/or generate a message alarm indicating that an unauthorized accessory has been attached.

In an alternate embodiment, we provide a method for detecting the number of times the blade or cutting accessory has been used.

### BRIEF DESCRIPTION OF THE DRAWINGS

FIG. 1 shows the Smart Shaver™ console 1, which is connected to its shaver handpiece 2, via a cable 3. A cutting accessory 4 is in this example connected to the handpiece 2. The Smart Shaver™ console 1 is further connected to an arthroscopy pump system 5 via control cable 6.

FIGS. 2A-2C comprise side cross-sectional views showing the basic components of the handpiece 2 of this invention. In FIG. 2A the handpiece 2 and the connecting cable 3 are shown. FIG. 2B is a cross-sectional view showing in further detail the components of the handpiece 2 of this invention. Encapsulated motor 21 is suspended with fixation units 23. The reactive rotation is picked up by load cells 22, which send signals to the shaver console 1 via the cable 3). The rotation of the motor axle is detected by Hall elements 24). Point 25 illustrates the coupling assembly to encompass the bore of the cutting accessory 4, in this case a burr. Said coupling assembly 25 also houses the transceiver 26 that transfers signals to or from the information carrier (not shown) in the cutting accessory 4. FIG. 2C shows an alternative embodiment for the handpiece of this invention. The motor is fixed in the shaver handpiece housing. A torque sensor 27 on the motor shaft by the coupling assembly signals torque value to the shaver console 1 via the cable 3.

FIG. 3A is a side view of a burr, which is an example of the cutting accessory 4 of this invention. Element 30 is the information carrier, in this example an RFID tag. FIG. 3B shows the burr when secured to the shaver handpiece.

FIG. 4 is a graph showing variations of torque 40 and utilization factor 41 of a shaver's cutting accessory over a time interval. The point 42 represents an accumulated utilization factor UF 1, which accounts for the torque and burr rotation speed integrated over time. The point 43 represents a second utilization factor, UF 2, which is the result of excessive torque and burr rotation speed integrated over time. Element 44 is the resulting Utilization Level. In this example two Utilization Factors are presented.

FIG. 5 illustrates another alternative for calculating and storing various Utilization factors for a particular cutting tool. Each block, A, B and C represents different Utilization Factors 50, 51 and 52 expressed as a % of a max Utilization Factor 53

FIG. 6 is a graph showing Utilization Level 44 on a time span of the life of the cutting accessory. Each step, 60, 61 and 62 represents different surgical procedures. On the third procedure, the Utilization Level reaches 100% at the line 63, and the cutting accessory has expired. The small steps show uses during the procedure. The Utilization factor is stepped up every time the tool is used during the procedure.

### DETAILED DESCRIPTION AND PREFERRED EMBODIMENTS

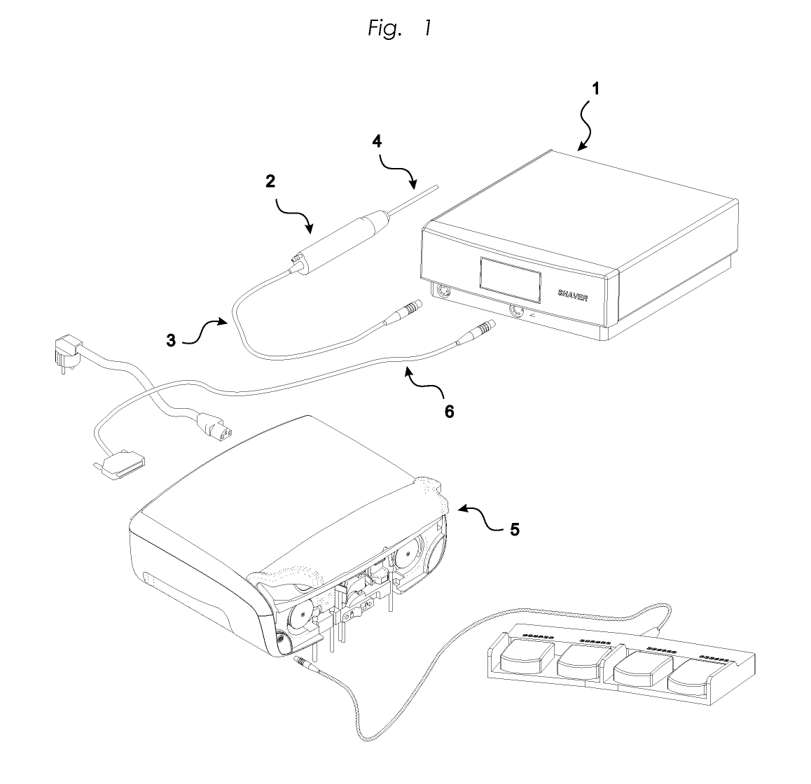

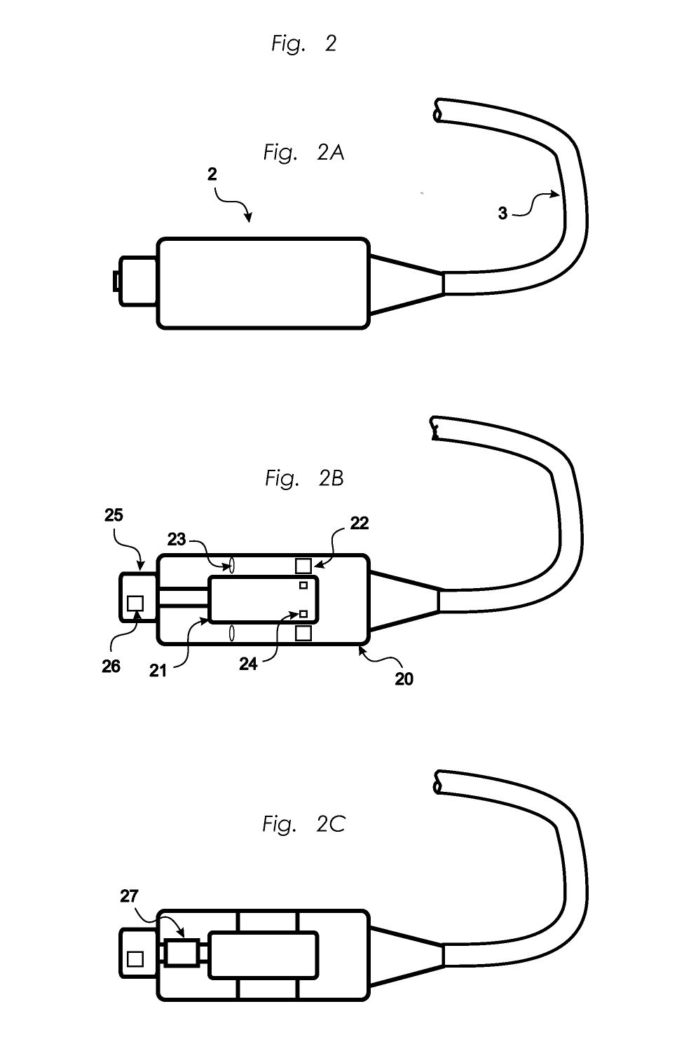

This invention concerns medical instruments, and particularly electrically powered surgical tools comprising a handpiece actuating removable rotary cutting accessories such as blades, burrs, edgers, resectors, planers, shavers, cutters, etc. As depicted in FIG. 2B, the handpiece 2 contains a motor 21 for driving the cutting and a coupling assembly 25 adapted to receive a proximal portion of the cutting device 4 (FIG. 1). The handpiece 2 usually includes an elongated housing, which distal end is formed with a bore for receiving the proximal end of the cutting accessory. The motor 21 is located inside the housing 20. A rotating output shaft, connected to the motor is disposed inside the bore. The coupling assembly 25 is arranged to the front end of the housing for removably holding the cutting accessory 4 to the handpiece 2. Further, the motor 21 is rotatably affixed to the housing 20 with at least one fixation 23. The fixation allows reactive rotation of the motor 21 in relation to the housing 20. Said reactive rotation is held back by at least one force sensor, sometimes also called load cell 22, FIG. 2A. The reaction force of the motor rotation is measured with the force sensor(s) 22. The reaction force is directly proportional to the forces to accelerate the motor shaft, coupling assembly 25 and the rotating parts of the cutting accessory 4 plus the torque 40 exerted by the motor 21 from load on the cutting accessory 4. A short time after power is applied to the motor, it reaches set speed, and no acceleration force is exerted on torque sensor(s) 27, located, in specific embodiments, on the motor shaft by the coupling assembly 25, FIG. 2C. Alternatively, they are situated in the motor itself or anywhere on the shaft between the motor and the cutting accessory. Some friction force may be present. Thus, when the motor has accelerated to set speed, the force measured by the force sensor(s) 22 is directly proportional to the load on the cutting accessory 4. The quality of the data obtained is considerably improved by the use of force sensors, directly measuring the torque exerted by the motor in the handpiece.

In preferred embodiments, we measure how much torque has been exerted and/or the amount of power consumed within an operative time interval. Workload history, workload distribution and run-time are accounted in a non-linear equation reflecting the actual wear of the cutting accessory. In alternative embodiments, the torque is measured by means of a torque transducer 27 on the motor shaft. This could for instance form a fitting between the motor 21 and the coupling assembly 25 for the cutting accessory. Both alternatives for measuring torque result in analog signals in the handpiece. These signals are amplified and fed via wiring from the handpiece to the console 1. This wiring is preferably a part of the cable 3 that delivers power from the console 1 to the shaver handpiece 2. In yet alternative embodiments, the torque can be calculated based on the electric current used to drive the motor 21 and the applied voltage. Furthermore, the rotational speed of the motor can be determined by rotation detectors, as further described below, and used together with the drive current and the applied voltage to calculate the torque at the motor and motor friction forces. The torque can further be measured by detecting the phase angle between the alternating current to the motor 21 and the signals from the Hall elements 24, as this phase angle increases with increased torque.

Consequently, power to actuate the handpiece comes from a control console 1 or external control module. The control console basically is an electrical power source for the shaver hand piece 2. The motor 21 in the hand piece may be a DC motor, meaning that the higher the applied voltage to the shaver handpiece, the faster the motor in it will run. More common is a three-phase motor. The control console will then deliver a voltage in three voltage phases, and as a rule also has a rotation speed feedback from rotation detectors. Such detectors typically comprise magnets affixed to the rotating part of the motor, and magnetic field sensors—typically Hall elements 24 affixed to the inside of the housing, FIG. 2B. The Hall element(s) 24 signal pulses to the shaver console 1 for every turn the motor makes. A pulse is sent every time a magnet moves passed through it, thus detecting a revolution. There may be several Hall elements and several magnets, sending pulses that indicate the phase angle of the motor. The pulse frequency indicates the rotation speed to the control console 1. For instance, if the number of revolutions per minute detected by the sensors is lower than that to correspond to the set speed, the power is increased to the motor and vice versa.

The control console 1 may be integrated with a pump system for this type of surgical procedure. For instance, a suction source 5, in most cases a pump for these procedures, is connected to the handpiece 2 (FIG. 1). In an arthroscopic surgical procedure, the body cavity is not opened so the surgical area is commonly made visible via the arthroscope. Detached tissue and debris is removed from the surgical site, through aspiration of the liquid in the joint via a liquid path through the cutting accessory and aspiration channel of the handpiece by means of the suction source. The irrigation liquid is as a rule saline and the pump is usually a peristaltic roller type pump.

The invention herein is based on our results showing that the torque at the motor more genuinely will reflect the actual load on the cutting accessory as compared with e.g. the amount of power consumed for driving the motor. The higher the accumulated workload, the more the cutting accessory has worn down by use. A relatively low but constant torque reflects a degree of wear that is specific for the type of accessory. Each product type—for instance a shaver burr—is subjected to wear under normal use and more intensively during abnormal use (excessive wear). The same torque for a so-called burr tool may mean lesser wear as compared with other tools. The tip of a cutting accessory may have a different diameter, number of cutting edges or be made of material with different characteristic. This wear may be non-linear to the current torque. For example, double torque may mean a quadruple wear or have a similar non-linear relation. The aforementioned torque levels may be established empirically in a lab environment by measuring the torque values of harsh use of the cutting accessory. It shall be noted that these levels are dependent on tissue properties, cutting accessory rotation speed, product type and run-time. Exceeding these levels characterizes an excessive wear factor.

The torque signals from force transducers 22 or specific torque transducer 27 on the motor or coupling assembly are received and calculated in the control console 1. Depending on torque values 40, a wear or utilization factor (UF) is calculated by the control console. Different utilization factors (UF) will depend on other variable factors such as torque peak values, number of rotations, number of sterilizations, product type and rotation speed. In combination, the sum of all utilization factors accumulated over time forms a degree of utilization (Utilization Level) that can be shown for the user on a display—such as a “bar graph” or a percentage of usage indicated on a display. Thus in specific embodiments the Utilization Level (UL) represents a cutting accessory's wear in % and is an indication of its actual remaining lifetime. Optionally an acoustic and/or light alarm will indicate if a high level of utilization has been reached; for instance 85%. This alarm level can be a warning level at one level, and an alarm at another level e.g. 95%. In another alternative, a lower warning level at first initialization can be used to warn against commencing a lengthy procedure such that to avoid having to replace the cutting accessory in the middle of that procedure. The process by which a device is used for the first time in a surgical procedure and hence data transfer takes place by the first time will be further referred in the present invention as first initialization.

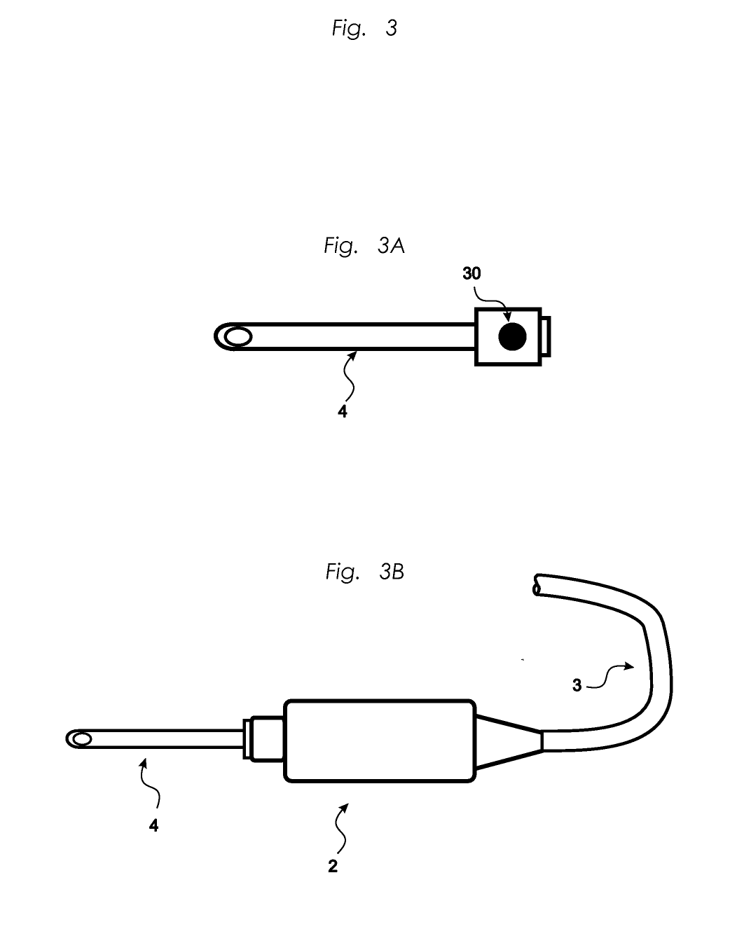

Data about the afore-mentioned variable factors is placed on at least one information carrier 30, which is fixedly attached to the cutting accessory 4, i.e. a burr, FIG. 3A. Said carrier may have the nature of non-volatile memory such as a RFID (Radio Frequency Identification Device) chip, smart card, memory card, sensors, magnets, coding bars, etc., that is adapted to deliver information to a reading element in the handpiece, and to provide the reading element with such information in a contactless fashion. Further, the control console 1 reads the information from the handpiece. The information carrier may also receive information from the control console via a transmitting element (not shown) in the handpiece. The reading element and transmitting element may be combined to a transceiver 26, FIG. 2B.

As previously explained, the handpiece 2 is connected to the console 1 by a cable 3. This provides power to the handpiece motor 21, signals actual motor rotation and exerted torque 40 to the console, and further transfers signals to/from the transceiver 26 from/to the console. The transfer of information between the information carrier and the reading element starts when a cutting accessory is connected to the system i.e. to the handpiece. The identification device of the invention herein comprises at least one reprogrammable information carrier 30 containing data comprising an ID number (namely serial number), type, code and product history e.g. number of accumulated rotations, number of uses, and accumulated torque used to drive the cutting accessory, etc.

A monitor in the control console 1 of the Smart Shaver™ system collects information from the handpiece 2. Normally, the type of information collected includes: a) how many rotations the shaver blade makes during an operation and b) how much torque 40 the handpiece motor 21 has used to drive the shaver blade during the same operation, c) torque peaks and; d) temperature in the motor. The information coming from the information carrier is processed by the system e.g. by the control console to calculate the different utilization factors; and is used to inform the user about the further utilization of the re-usable blade. Accumulated data from previous use of the specific shaver blade, in combination with actual online data are thus utilized in the algorithm for calculating the utilization level (UL), displayed on the presentation unit of the console. Instead or in addition to displaying information relating to the utilization level of the cutting accessory on a presentation unit, the control console 1 may be configured to limit or prevent further operation of the cutting accessory in dependence of the calculated utilization level. For example, the control console 1 may be adapted to limit or prevent further operation of the cutting accessory when the utilization level exceeds a predetermined threshold value. In one embodiment, the control console 1 may be configured to prevent the rotational speed of the motor 21 from exceeding a certain maximum value when the utilization level exceeds a predetermined threshold value. The maximum value for the rotational speed of the motor may be set to a predetermined positive RPM value meaning that further operation of the cutting accessory is limited, or be set to zero meaning that further operation of the cutting accessory is prevented. In another embodiment, the control console 1 may be configured to set a maximum time during which the cutting accessory can be operated after the predetermined utilization level threshold value has been exceeded. In yet other embodiments, the control console may be configured to prevent further operation of the cutting accessory by preventing reading of information from the information carrier 30 of the cutting accessory. This may be achieved by the control console by sending information to the information carrier making the information carrier unreadable, thereby preventing the control console from recognizing and accepting the cutting accessory. It may also be achieved by the control console by generating an over current that destroys the information carrier 30 and so prevents further operation of the cutting accessory.

Information about the number of rotations the motor makes is collected from the servo drive module in the console. This data is at hand for the system, as the rotation normally is detected by sensors in the motor 24. For every revolution the motor makes pulses are sent by these sensors. Theses pulses are transferred to the control console via the cable 3. In some cases the control console can acquire rotation data from the servo module. The number of motor revolutions is not always the same as those of the cutting accessory because there often is a gearbox connected to the motor. For instance, if the gearbox has a ratio of 1:8, and the motor runs at 64,000 revolutions per minute (rpm), the cutting accessory rotates at 8,000 rpm.

Periodically, after each use of the cutting accessory i.e. the shaver blade, actual accumulated information, comprising both historical data and data collected during a surgical operation, are sent to the information carrier such that the tag is always updated with actual accumulated data for the particular blade. For instance, the data content in the information carrier can be updated every time power is released from the motor. Thus the information carrier acts as a read/write memory presenting data that can be overwritten and reprogrammed by the system.

One or several information carriers may also include data describing the intended use of the blade i.e. functional data model or type, the speed range within which the cutting device is to operate, sterilization data, i.e. sterilization date, number of sterilizations, etc. This information may be used to restrict the functionality of the system, for example as follows:

The unique blade code will be checked by the system. Thus if an unauthorized blade is connected, the user will be alerted that an unauthorized device is in use (a warning in the operating manual will clarify the consequences if an unauthorized device is used).

In particular embodiments the apparatus may be programmed to even shut down if the article is not approved, therewith avoiding an accident. The stop/alarm signal might also be sent in those cases when the manufacturer has withdrawn the article batch number/lot number, or the lease or service period has expired.

Selectively coding data for a specific blade type (REF number) is also used by the system to determine the speed range within which the instrument is to operate and the default settings for such blade.

In certain particular embodiments, the coding data is self-identification data ordering for operating a parameter set that is preferred by the user. For example, if a surgeon has specific set up preferences for the suction pump i.e. pressure values, flow values, blood and debris detection sensitivity, these would be automatically set upon connection of the cutting accessory to the hand piece. Further, such setup of operating parameters may also describe the generally accepted setup for a specific surgical procedure with regard to power supply, voltage, current parameters, etc. for the shaver system or any other system that is incorporated in the surgical setup, such as an electrosurgical unit or a video or imaging system. In other specific embodiments the data from the information carriers may carry setup values for ACL (anterior cruciate ligament) surgery, small joint surgical procedures or pressure and flow values for meniscus resectomies. Such setup values can be settings for irrigation pump pressure and flow settings for aspiration.

Data on the number of uses may be calculated in the following way: The control console has a time and date functionality incorporated that identifies actual date and time. Technically this is referred to as a “real time clock”, that is commonly an integrated circuit that can deliver numbers corresponding to the current day, month hour, etc. Information on date and time is gathered in the information carrier of the cutting accessory every minute during the time cutting accessory is connected to the handpiece. Briefly, once the console has registered first initialization or a new initialization, the logistics that determines if the accessory has been used also determines if dates and time values registered on the information carrier correspond to previous uses. Each subsequent initialization after the first initialization, will be referred in this application, as second, third, etc. initialization. For example, if the last registered date was on the day before a new and third initialization, the accessory has been used, and historical and operational data on previous processes is stored onto the information carrier. In those situations where the console cannot read data from the information carrier, it can be assumed that the equipment has been disconnected. Those disconnections periods are themselves registered with identification number in the memory of the control console. If disconnection time exceeds that of a normal surgical procedure i.e. one hour, then the cutting accessory has been used, which is another means to determine use.

The date and time system functionality described above may optionally be a general asynchronous timer. In this case, date and time are numerical values that do not necessary relate to a specific time, but rather usage time of the device.

Data may be displayed for the user throughout the whole surgical procedure from first initialization of the Smart Shaver™ system or on certain pre-determined occasions.

In alternative embodiments of this invention, when the article has passed the manufacturer's recommended max usage or the calculated wear values surpass a pre-established maximum limit, the apparatus may be programmed to shut down or to send a warning alarm. In some cases, though, the article i.e. the cutting accessory can be allowed to be connected to an apparatus several times, although within a limited time period, if the surgeons so wish. As the system measures and stores information regarding utilization of the accessory in specific surgical operations, number of uses, accumulated workload, recommended and calculated max usage, etc.; it may also be programmed to give permission or to deny a further use of a cutting accessory due to security reasons. Our system enables you to reprogram and change the allowed usage interval range of the calculated wear factor in advance, to also fulfill very specific health—risk regulations.

As to further discussion of the manner of usage and operation of the present invention, the same should be apparent from the above description. Accordingly, no further discussion relating to the manner of usage and operation will be provided. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### EXAMPLES

### Example 1

### Calculation of Utilization Level Considering Different Variable Factors

The invention herein teaches an algorithm for accurately calculating the level of utilization or wear, UL. Different factors such as the sensitivity to torque, k, of each accessory, will add further accuracy to our method. The value of k differs for each type of cutting tool ((burr, aggressive burrs, meniscus cutters).

UF 1 is the first Utilization Factor, wherein:

UF 1=_{begin}∫^{end}k₁×m×r(dt).

k₁ is the sensitivity to torque
_{begin}∫^{end} is the integral of the time the cutting accessory starts and ends rotation
m is the torque [Nm]
r is rotation speed [rpm]
dt is the accessory running time in the specific interval

The value of k₁ is directly proportional to wear. A high constant value for a specific type of cutting tool will mean rapid wear at a certain torque speed.

Each time interval, and hence each Utilization Level, UL, will consider several UF values e.g. UF1, UF2, UF3 and UF4, which are sensible or account for different variable factors. Consequently, a second Utilization Factor might be calculated as follow.

UF 2=_{begin}∫^{end}×k₂×(1+m)^{y}×r(dt), wherein:

k₂ and y determines the particular cutting accessory's sensitivity to high torque levels.

UF 3 reflects the third Utilization Factor, wherein:

UF 3=_{begin}∫^{end}k₃×r(dt)

k₃ determines the particular cutting accessory's sensitivity to rotation; not in respect of load or torque.

It should be noted that constants k₁, k₂, k₃ and k₄ are all dependent on the type of cutting accessory. However their value can vary when different utilization factors, each dependent of different variable factors, are calculated for a same tool.

UF 4 reflects the fourth Utilization Factor, wherein:

UF 4=k₄×n

k₄, is a quality factor that specifies how prone the cutting accessory is to deterioration due to number of uses.

The accessory is sterilized prior to every procedure, which contributes to the value of this constant. The number of uses is calculated according to the time logic described above, and the Utilization Factor UF4 applies for every surgical procedure.

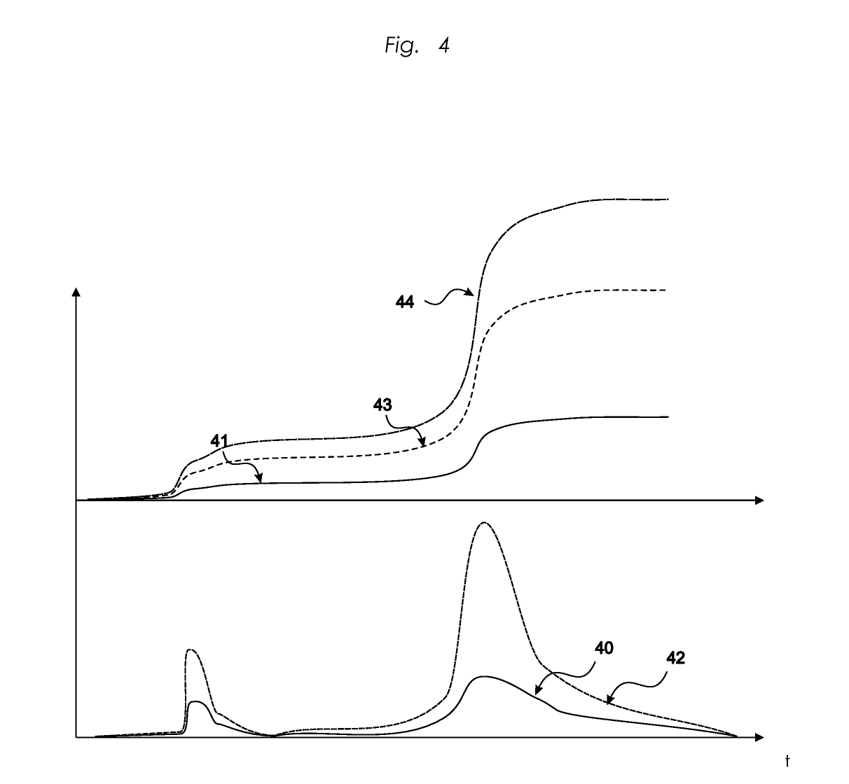

The accumulative workload in each interval is presented as a total accumulative and integrated utilization level (UL) 44, which thus is considered the sum of several Utilization Factors (UF 1+UF 2+UF 3+UF 4) for every procedure or part thereof (FIG. 4).

The degree of wear of the cutting accessory is expressed as the utilization in proportion to how much it can be used, where 100% means that the cutting accessory has been exhausted from use.

This limit of utilization is calculated as follow: UL=UF1+UF2+UF3+UF4 [%]

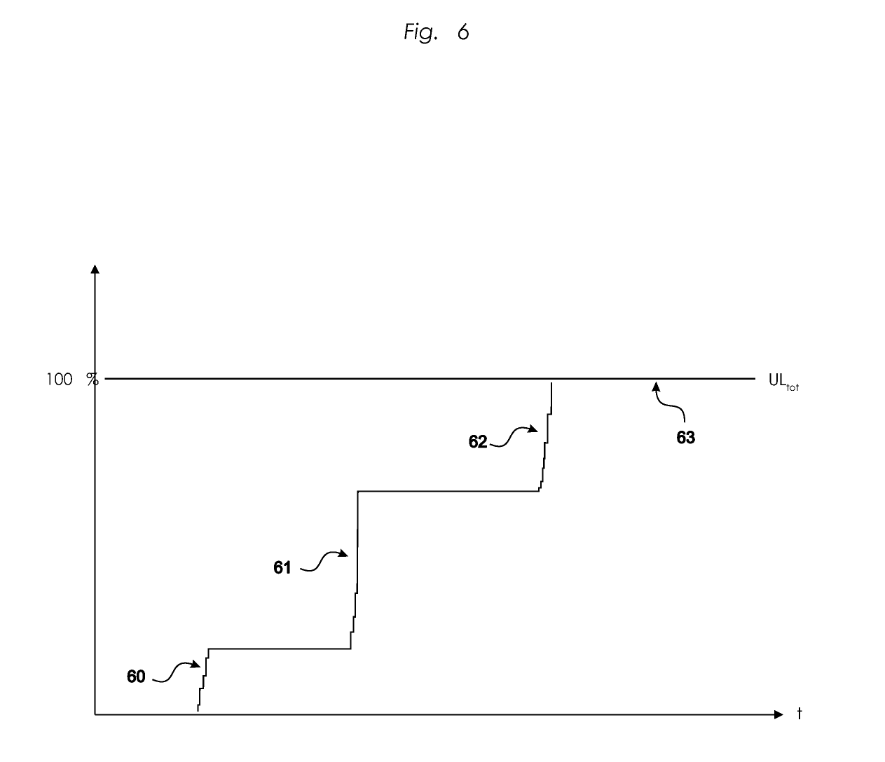

The Utilization Levels is stepped up for every surgical procedure, and the 100% ceiling can be reached during the first, second or any other procedure, depending on how excessively the surgical tool is used during the procedures (FIG. 6).

The accumulated Utilization Level for several surgical procedures can be expressed as: ULₜₒₜ=UL₁+UL₂+UL₃+UL₄ [%]

Where the total Utilization Factor ULₜₒₜ is the sum of the contributing Utilization Factors from each procedure (here 1,2,3 and 4).

### Example 2

The general methodology described in Example 1 is hereby employed for calculating the Utilization Level when using a “meniscus cutter” during two operational procedures. This tool has a steel quality of DIN 1.4305, a material well suitable for the purpose. The specific material and product type determine the value of each parameter in the Utilization Level algorithm:

k_{1′}=0.0001
k₂=0.00001
y=3.0
k₃=0.000002
k₄=2

The initial run time (dt1) of the meniscus cutter is in this example 20 seconds with a moderate low average torque (m) of 0.06 Nm, representing soft tissue. Thereafter a second run (dt2) of only one second has a high torque of 5 Nm, as the tool is pressed hard against bone material. The rotation speed is 4 000 rpm.

For the first 20 second run:

UF1=0.0001×0.06×4 000×20=0.48

UF2=0.00001(×(1+0.06)^{3.0}×4 000×20=0.95

UF3=0.000002×4 000×20=0.16

UF4=2

The Utilization Level that applies to this 20 second run is calculated as follow:

UL=0.48+0.95+0.16+2=3.59%

In the second run of one second, the torque is relatively high, and the wear is considerably higher, although the application time is short:

UL 1=0.0001×5×4 000×1=2.00

UF2=0.00001(×(1+5)^{3.0}×4 000×1=8.64

UF3=0.000002×4 000×1=0.08

UF4=0

UF4 equals 0 in this running interval because this run has been made during the same surgical procedure as the first one.

Thus, UL=2.00+8.64+0.08+0=10.72

In this example, the meniscus cutter is only used twice during the surgical procedure.

ULₜₒₜ=3.59+10.72=14.31%

### Example 3

### Wear Calculations Using Max Torque as the Only Variable Factor

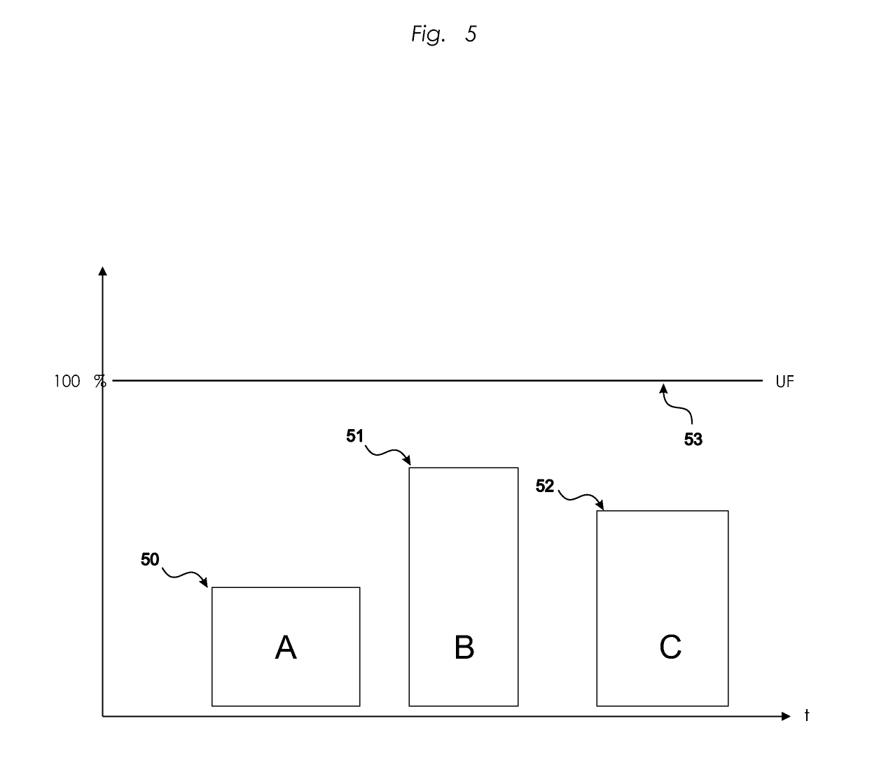

As depicted in FIG. 5, the various levels of torque can in an alternative calculation method to the above be classified into different torque categories. In this example, if the torque is in the range 0-0.7 Nm it falls into category A, if it is from 0.7 to 2 Nm it falls into category B; and if over 2 Nm it falls into category C. A surgical procedure in which the torque is in category A during the whole process, will have an Utilization factor UFA that is the result of multiplying the time the torque falls within this category by 1.

For category B and C the Utilization Factor is obtained by multiplying the torque by 1.5 and 5, respectively. The torque falls under category A in those cases when the cutting accessory is used for cutting soft tissue; in category B for hard tissue; and in category C for cutting bone material. The time each torque category has occurred adds to the representative Utilization factor A, B or C respectively.

In FIG. 5, each block, A, B and C, 50, 51 and 52 represents these different Utilization Factors, and are expressed in % of the max Utilization 53 allowed for the particular cutting tool.

1. A method for determining a utilization level indicating a degree of utilization of a re-usable cutting accessory for endoscopic surgery, removably connectable to a handpiece comprising a motor driving the cutting accessory during surgical procedures, wherein the determination of the utilization level of the cutting accessory involves calculation of at least one utilization factor that is calculated based on a torque measured when said cutting accessory is driven by said motor.

2. The method according to claim 1, wherein said at least one utilization factor is calculated based on the torque and the rotation speed of said motor.

3. Method according to claim 1, comprising the step of calculating a first utilization factor indicative of an accumulated torque used to drive the cutting accessory.

4. Method according to claim 3, further comprising the step of calculating a second utilization factor indicative of torque peaks over a set value.

5. The method according to claim 4, further comprising the steps of calculating a third utilization factor indicative of the number of rotations of the cutting accessory, and a fourth utilization factor indicative of the number of uses of the cutting accessory.

6. The method according to claim 1, wherein the at least one utilization factor is calculated using a sensitivity factor that differs for different types of cutting accessories.

7. The method according to claim 1, wherein the determination of the utilization level comprises the steps of:
calculating a plurality of utilization factors, and
determining the utilization level from the sum of all utilization factors accumulated over time.

8. The method according to claim 1, wherein the utilization level is determined as an accumulated utilization level from the sum of a plurality of utilization levels determined for different surgical procedures.

9. The method according to claim 1, further comprising the step of showing the utilization level of the cutting accessory to a user on a display.

10. The method according to claim 1, further comprising the step of generating an alarm when a high level of utilization of the cutting accessory has been reached.

11. The method according to claim 1, further comprising the step of sending, after each use of the cutting accessory, actual accumulated information comprising both historical data and data collected during a surgical operation to an information carrier of the cutting accessory, such that the information carrier is always updated with actual accumulated data for the particular cutting accessory.

12. A control console for determining a utilization level indicating a degree of utilization of a re-usable cutting accessory for endoscopic surgery, removably connectable to a handpiece comprising a motor driving the cutting accessory during surgical procedures, wherein the control console is configured to obtain information indicative of a torque measured when said cutting accessory is driven by said motor, and to determine the utilization level of the cutting accessory by calculating at least one utilization factor based on said torque.

13. The control console according to claim 12, further being configured to receive signals indicative of the rotation speed of said motor from a sensor of said handpiece, and to calculate said at least one utilization factor based on the torque and the rotation speed of said motor.

14. The control console according to claim 12, wherein the control console is configured to calculate a first utilization factor indicative of an accumulated torque used to drive the cutting accessory.

15. The control console according to claim 14, wherein the control console is further configured to calculate a second utilization factor indicative of torque peaks over a set value.

16. The control console according to claim 15, wherein the control console is further configured to calculate a third utilization factor indicative of the number of rotations of the cutting accessory, and a fourth utilization factor indicative of the number of uses of the cutting accessory.

17. The control console according to claim 12, wherein the control console is configured to calculate the at least one utilization factor using a sensitivity factor that differs for different types of cutting accessories.

18. The control console according to claim 12, wherein the control console is configured to determine the utilization level by calculating a plurality of utilization factors, and determining the utilization level from the sum of all utilization factors accumulated over time.

19. The control console according to claim 12, wherein the control console is configured to determine the utilization level as an accumulated utilization level from the sum of a plurality of utilization levels determined for different surgical procedures.

20. The control console according to claim 12, wherein the control console is configured to show the utilization level of the cutting accessory to a user on a display.

21. The control console according to claim 12, wherein the control console is configured to generate an alarm when a high level of utilization of the cutting accessory has been reached.

22. The control console according to claim 12, wherein the control console is configured to send, after each use of the cutting accessory, actual accumulated information comprising both historical data and data collected during a surgical operation to a transceiver of the handpiece for further transmission to an information carrier of the cutting accessory, such that the information carrier is always updated with actual accumulated data for the particular cutting accessory.

23. The control console according to claim 12, wherein the control console is configured to obtain the information indicative of said torque through the reception of torque signals from a transducer of said handpiece.

24. A system for determining a utilization level indicating a degree of utilization of a re-usable cutting accessory for endoscopic surgery, comprising:
a control console, and
a handpiece connectable to the control console and to the re-usable cutting accessory, wherein said handpiece comprises a motor for driving the cutting accessory during surgical procedures, and a transducer for measuring a torque when the cutting accessory is driven by the motor, and transmitting torque signals indicative of said torque to the control console,
wherein the control console is a control console according to claim 12.

25. A re-usable cutting accessory for endoscopic surgery, comprising an information carrier acting as a read/write memory that can be overwritten by a control console to which the cutting accessory is connectable via a handpiece, wherein the information carrier is configured to receive, after each use of the cutting accessory, information on accumulated torque measured during use of the cutting accessory from a transmitting element in the handpiece, and to store said information for subsequent use by the control console to calculate said at least one utilization factor.
